(19) United States
(12) Patent Application Publication
(10) Pub. No.: US 2011/0293679 A1
(43) Pub. Date: Dec. 1, 2011

(54) METHOD FOR COATING MICROSPHERES ONTO A FLEXIBLE MATERIAL

(76) Inventors: René Chelle, Grepiac (FR); Urbain Makoumbou, Frouzins (FR); Jean-Pierre Lautier, Labege (FR)

(21) Appl. No.: 13/147,212

(22) PCT Filed: Jan. 27, 2010
(86) PCT No.: PCT/FR10/50127

§ 371 Date: Aug. 1, 2011

(30) Foreign Application Priority Data
Feb. 9, 2009 (FR) 0950787

(51) Int. Cl. A61K 8/02; B32B 3/26; B05D 3/02; A61Q 19/00; A61K 9/70; A61P 29/00; A61P 17/00; B32B 5/16; B32B 5/02

(52) U.S. Cl. 424/401; 428/316.6; 427/2.31; 427/180; 442/96; 442/59; 424/402

(57) ABSTRACT

The invention relates to a method for coating polymer microspheres laden with one or more active substances onto a flexible material without melting, comprising the following steps: a) selecting a porous crosslinked polymer; b) incorporating a liquid lipophilic composition at room temperature and containing at least one active substance into the polymer by heating at a temperature higher by 1 to 5° C. than the glass transition temperature of the polymer in order to obtain laden microspheres; c) placing the laden microspheres on the flexible material; d) heating the flexible material with the laden microspheres at a temperature higher by 1 to 10° C. than the polymer melting temperature; e) cooling the flexible material impregnated with the laden microspheres. The coated flexible material of the invention is laden with one or more active substances having a therapeutic, dermatological cosmetic or olfactory effect and the diffusion is controlled on the basis of the polymers used for making the microspheres and on the nature of the active substances.

The present invention relates to the application of material onto a flexible material. It relates more precisely to the coating without melting, that is to say the placement in intimate contact by fixing, of fibrous or porous media such as textiles and non-woven materials by polymer microspheres laden with one or more active substances.

Generally, the application of material onto a flexible material is accomplished by coating or laminating. The coating process consists in principle of coating a flat surface. There are several benefits in coating a material such as a textile. It can impart different properties to the fabric such as protection against liquids, stains or fire, and it can help to strengthen the material or modify its feel. The fabric, once coated, can be textured to impart different finishes thereto (matt, glossy, imitation leather, etc.). The majority of known coating applications are carried out with the object of strengthening, modifying or adapting the physical and mechanical characteristics of a textile or a flexible material in order to provide a specific effect or use (Noel PONTHUS: “Industrial Textiles”, WO/2008/080867: “Method for preparing a woven, knitted or non woven fibrous substrate coated on one or both sides with at least one layer of reinforced elastomeric silicon”, WO/2007/112982: “Process for coating a textile surface,” WO/2007/039763: “A liquid silicone rubber composition for textile coating,” ESMERY CARON Structures: Textile architecture, Tents and Tarpaulins, CAPLP2/EXTERNAL COMPETITION: MAINTENANCE OF TEXTILE ARTICLES: SESSION 2003).

Irrespective of the polymer used: silicone, PVC, acrylic, polyurethane, etc., the coating is generally in the form of a liquid or a paste that is spread then fixed by heating on the surface of the substrate (FR 2851265, A. Ponche and D. Dupuis: Rheology, Vol. 2, 39-45 (2002): “Relationship between rheometry and structure in the case of suspensions of titanium dioxide in polymer solutions”). When the polymer is solid, an operation of gelification (melting/liquefaction) takes place with in-situ polymerisation (WO/0011061 (A1): “Method for coating textile”, ARKEMA: LACOVYL®: Coating—Application of the process). The operations of solubilisation or gelification of the different composites call for the use of so-called coating aids and volatile solvents which must be subsequently removed and which are thus a source of environmental problems (179, UBA (Federal Office for the Environment), 2001: Coating and laminating).

The polymer most commonly used for coating textiles is polyvinyl chloride (PVC) (ITF—Research and Development: “Materials and Technologies” (1999), 63, GuT/ECA, 2000: “Back coating of carpets.” Different types of PVC coating are thus described such as clear PVC coating (inside or outside tablecloths, table sets), white PVC coating (draw sheets), clear or coloured PVC coating with grain (leather goods, bags, cases), back coating (upholstery), PVC coating for industrial textiles. In the latter case, the typical coating is composed of 80% PVC, 13% polyurethane and 7% other polymers and auxiliary products.

The polluting elements associated with the presence of auxiliary products are generally carcinogenic residual monomers such as acrylonitrile, vinyl chloride, acrylamide, 1,3-butadiene and vinyl cyclohexene, as well as decomposition products originating mainly from additives.

Conventional devices used in coating processes include a cylinder (transfer cylinder) or a squeegee (blade) that moves tangentially to the surface of the material to be coated in order to apply a defined amount (grams or cm³ per m²) of coating, in the case of direct coating. The coating can also be accomplished indirectly, in which case it is referred to as transfer coating. In this case the coating is first applied onto another substrate, then the coated face is laminated onto the final material (usually fragile).

Other types of coating are also known in which different polymers and even metals are applied onto fibres or textiles. The term “coated textile” is understood to mean a textile substrate with a polymer resin added thereto in an amount ranging from a fraction of the weight of the textile to several times its weight. All textile materials can potentially be used, however the compatibility between the fibre and the coating must be taken into account. The coating formulations in these cases are based on polyvinyl chloride, polyurethane, acrylic and natural or synthetic elastomers. The addition of plasticisers, mineral fillers or other auxiliary materials is necessary for fixing the polymers and also serves to impart additional properties to the material for the purposes of a given process or end use.

It is possible, for example, to carry out a complexing process whereby films, foams or membranes (microporous, polyurethane (PUR) or polytetrafluoroethylene (PTFE)) are laminated onto a textile substrate, thereby imparting to the complex the functions of a breathable barrier or waterproofing.

Mention may also be made of pre-impregnated materials in the case of textile structures treated with a non-crosslinked thermosetting resin or a thermoplastic resin. These textile structures are pre-impregnated by solution processes, melting, powder-coating, hybridation or transfer processes. During a heat treatment, the pre-impregnated material is shaped in a mould.

In a general manner, textile coating and laminating products can be divided into five main families according to their chemical composition.

Coating materials in powder form based on:
polyolefins (especially polyethylene)
polyamide 6,
polyamide 6.6,
copolyamides,
polyester,
polyurethane
polyvinyl chloride,
polytetrafluoroethylene.

Coating products in paste form based on the polymers mentioned hereinabove and also containing additives such as:
dispersants (surfactants, often alkylphenol ethoxylates),
solubilising agents (glycols, N-methylpyrrolidone, hydrocarbons),
foaming agents (mineral oils, fatty acids, ammonium salts of fatty acids),
softeners (particularly phthalates, sulfonamides),
thickeners (polyacrylates),
ammonia.

Coating products in the form of polymer dispersions (aqueous formulations) containing approximately 50% water, based on:
polymethacrylate of (butyl, ethyl, methyl, etc.)
polyacrylic acid,
polyacrylonitrile,
polyacrylamide
1,3-polybutadiene,
polystyrene,
polyurethane
polyvinyl chloride,
polyvinyl acetate,
and the copolymers and polymers mentioned hereinabove.

Additives are also present, as in paste coatings.

Coating products in the form of melamine resins, produced by the reaction of melamine and formaldehyde and by subsequent etherification mainly with methanol in an aqueous medium (water content from 50 to 70%).

Coating products in the form of polymer dispersions (formulations based on an organic solvent), based on polyurethane and silicones dispersed in an organic solvent.

In all cases, irrespective of the nature of the coating product, a heating step is necessary for the application of the polymer on the substrate. The purpose of this step is to bring the polymer to an advanced state of melting so that the internal structure of the polymer is altered enabling an intimate bond to be formed with the fibres of the substrate. It is carried out in ovens or tunnel kilns well known to the person skilled in the art.

Depending on the type of coating products, the environmental problems associated with emissions of toxic products into the air are not the same. They can occur directly during the use of the polymer itself as in the case of coating materials in powder form using polyamide 6 and its copolymers (the residual monomer ε-caprolactam is released at normal process temperatures).

They can also occur indirectly, but unavoidably, through the use of additives whose presence is necessary, as in the case of coating products in paste form. The toxic products released in this case are mainly:
fatty alcohols, fatty acids, fatty amines, originating from surfactants,
glycols originating from emulsifiers,
alkyl phenols originating from dispersants,
glycols, aliphatic hydrocarbons, N-methylpyrrolidone hydrotropic agents,
aliphatic hydrocarbons, fatty acids/salts, ammonia originating from foaming agents,
phthalates, sulfonamides/esters originating from softeners/plasticisers
acrylic acids, acrylates, ammonia, aliphatic hydrocarbons originating from thickeners.

With regard to coating products in the form of polymer dispersions (aqueous formulations), the constituents that are responsible for emissions into the air are dispersants, the residual constituents of polymerisation (especially t-butanol used as a catalyst in radically initiated polymerisation reactions) and monomers resulting from incomplete reaction during polymerisation. The latter are particularly important in the context of ambient air pollution in the workplace and odours. They include:
acrylates such as acrylic acid, butyl acrylate, ethyl acrylate, methyl acrylate, ethylhexyl acrylate and vinyl acetate,
carcinogenic monomers such as acrylonitrile, vinyl chloride, acrylamide, 1,3-butadiene and vinyl cyclohexene.

Vinyl cyclohexene is not often identified in the gaseous emissions. However, it is always formed (2+2 cyclo-addition products) when 1,3-butadiene is used. Acrylamide in the gaseous emissions is often associated with emissions of formaldehyde (reaction products of methylol-acrylamide).

Coating products in the form of melamine resins may contain considerable amounts of free formaldehyde and methanol. During their application, the crosslinking reaction of the resin with itself or with the fabric (for example cotton) is initiated by an acid catalyst and/or temperature, releasing stoichiometric quantities of methanol and formaldehyde.

Coating products in the form of polymer dispersions (formulations containing organic solvents), although uncommon in the textile finishing industry, require the use of equipment for treating gaseous emissions including thermal oxidation or adsorption onto activated carbon. Indeed, this process requires passage through an oven to polymerise the composite and eliminate volatile solvents before cooling and winding.

It is apparent therefore that, irrespective of the family of coating products used, a number of toxic compounds are emitted directly or indirectly during coating processes conventionally used.

The object of the present invention is to overcome the drawbacks of the prior art and to provide a method of coating flexible, non-woven materials or textiles by thermal application of polymer microspheres laden with one or more active substances, without any emission toxic to humans or the environment occurring during the operation. This result is obtained by the development of a method for fixing certain polymers selected for their physicochemical, structural and non-polluting properties, laden with active substance, in the form of microspheres, to the fibres or pores of the substrate, without resorting to the use of auxiliary products leading to the emission of toxic products into the air.

This technique makes it possible to impart active properties to the flexible material that is coated on completion of the method of the present invention, by virtue of the different properties of the active substances incorporated into the polymer microspheres. In the remainder of this description, the term “active substance” will be used in the context of the invention in the singular for convenience of wording, but will include in its scope a single substance or composition as well as several active substances or compositions mixed between them within the same microsphere, or mixed by the association of several microspheres each laden with one active substance.

The activity imparted to the flexible material via the laden microspheres results from the release of the active substance stored within the microspheres by diffusion through the microporous network thereof. The principle of the invention effectively lies in preserving the porous structure of the microspheres, despite the heat treatment that is applied to the polymers constituting the microspheres during the coating process. In a surprising manner, through the choice of certain specific polymers and in a precise range of temperatures, it was found that it was possible to obtain an intimate and solid fixing of the loaded microspheres to the fibres or pores of the flexible material by thermal application, without using auxiliary coating or plasticising products, while at the same time preserving the structure of the porous network of the microspheres.

The method of the present invention provides an activated flexible material offering the user an alternative to:
taking certain active substances conventionally via the oral route which poses a considerable number of disadvantages such as the hepatic first pass and the series of effects associated therewith,
the topical route which has the disadvantage of having to apply an ointment or gel containing the active substance several times a day, often with the considerable drawback of not having a suitable place in which to perform the application discreetly and correctly.

By placing the flexible material treated according to the method of the invention in contact with the skin, the user benefits from a continuous and prolonged release of the active substance stored in the microspheres which impregnates the fibres of the fabric. In the case of active substances such as active ingredients with therapeutic effect, this has the advantage of facilitating the treatments, not only in terms of administration and release of the active ingredient to the target area, but also in terms of diffusion and regularity of the doses that the user is required to take.

More precisely, the present invention relates to a method for coating polymer microspheres laden with one or more active substances onto a flexible material without melting, characterised in that it comprises the following steps:

a) selecting a porous crosslinked polymer,
b) incorporating a lipophilic composition liquid at room temperature and containing at least one active substance into the polymer by heating at a temperature higher by 1 to 5° C. than the glass transition temperature of the polymer in order to obtain laden microspheres,
c) placing the laden microspheres on a flexible material,
d) heating the flexible material with the laden microspheres at a temperature higher by 1 to 10° C. than the polymer melting temperature, without reaching the molten state of the polymer,
e) cooling the flexible material impregnated with the laden microspheres.

The nature of the polymers used to form the microspheres is crucial to the proper conduct of the method according to the invention. Specifically, they must have a reticular structure in order to retain the active substances associated therewith and thus be capable of storing them so as to form the microspheres. They must also have a porous network characterised by a plurality of interconnected micropores to allow the diffusion of the active substances after the process itself of coating the target substrate, to allow release into the outside environment and/or skin of the user who is wearing the substrate.

In the context of the invention, the microspheres are composed of a single type of porous crosslinked polymer. Preferably, the porous cross-linked polymers selected to form the microspheres are polyamides, polyether block amides or ethylene vinyl acetates. Several microspheres composed of different polymers selected from the aforementioned porous crosslinked polymers can be mixed. On an indicative basis, such microspheres have a diameter of between 5 and 200 μm, with a heterogeneous filled internal structure, consisting of a plurality of micropores interconnected to each other and having a diameter of about 0.1 to 10 μm.

There are known processes for coating textile fibres involving the use of microcapsules. The microcapsules may be composed of a wide variety of polymers and are characterised by a hollow structure enveloping in a membrane-like manner the active composition incorporated into the core of the microcapsule. The processes for manufacturing and charging the microcapsules are complex and costly. It is difficult to store large amounts of active composition within the microcapsules. Moreover, the stored composition is not released progressively, it is released en masse during the breakdown of the membrane forming the outer envelope of the microcapsule by mechanical rupture or chemical breakdown in a solvent such as water. Generally these microcapsules are not fixed directly to the substrate and require the use of binders or other auxiliary products. A step of heating the coating polymer is performed at high temperatures to improve the fixing of the microcapsules by melting onto the fibres of the substrate which can cause degradation of the stored active composition and a premature release of the latter by rupturing the wall of the microcapsule. In some cases the binder used mixes with the polymer matrix of the microcapsule (fixing on the substrate), which causes the microcapsule to become impermeable and slows down the diffusion of the remaining active composition. This type of fabric coated with microcapsules is ultimately shown to be ineffective in terms of diffusing the stored active composition and in terms of resistance to washing given the fragility inherent in the structure of the microcapsules and their poor fixing to the fibres of the substrate.

The active substances that can be used in the method of the invention are constituted by all kinds of compounds having an effect of a therapeutic, dermatological, cosmetic or olfactory nature. In order to facilitate their incorporation into the porous crosslinked polymers constituting the microspheres, the active substances are associated with lipophilic compositions that are liquid at room temperature. The lipophilic nature of the composition not only facilitates the incorporation of the active substance within the microsphere, but also improves the release thereof from the micropores, once the microsphere is fixed to the fibres or pores of the substrate, and improves the gradual diffusion in contact with the skin by virtue of the lipophilic interactions resulting from the sebum.

Preferably, the active substance is selected from molecules with the following type of action:
therapeutic (i.e., analgesic, anti-inflammatory, veinotonic, etc.);
dennatological-cosmetic (i.e., slimming-lipolytic and/or draining, moisturising, soothing, anti-ageing, antioxidant and/or restructuring and/or repairing, clarifying, heavy legs);
olfactory (i.e., anti-stress, deodorising/odorising, attractant and/or repellent).

It is associated with a lipophilic composition liquid at room temperature which can be formulated from essential oils, natural or synthetic essences, natural or synthetic substances in liquid form or dissolved in a lipophilic medium.

The incorporation itself of the active substance associated with the lipophilic composition liquid at room temperature within the microsphere is accomplished by simple heating of the porous crosslinked polymer at a temperature higher by 1 to 5° Celsius, preferably 3° Celsius, than the glass transition temperature of the said polymer to facilitate incorporation into the polymer without denaturing the structure of the microporous network thereof. The molten state of the polymer is not reached, the properties of the active substances are preserved. No technical plasticising additive is used. This step is performed in a sufficiently short time so as not to impair the physicochemical properties of the polymer; it will be of less than 5 minutes duration, preferably 3 minutes.

The active substance(s) is (are) incorporated into the porous crosslinked polymer in a weight ratio representing 1 to 40% by weight of the resulting laden microsphere. Several active substances of different types can thus be incorporated into one type of microspheres. The amounts of active substance stored in the microspheres are greater than in the charging of the microcapsules described in the prior art.

The next step involves placing the microspheres laden with one or more active substances onto the flexible material. To do this, the microspheres are distributed manually or automatically on the surface of the flexible material. The latter can be first disposed in any type of industrial apparatus for putting the coating process into effect in an industrial manner in order to obtain high production outputs such as conveyors or other form of automated line. It is possible to modulate the rate of diffusion of the stored active substances by distributing high concentrations of microspheres on the flexible material.

The flexible material is comprised of a fibrous or porous substrate. It includes a plurality of fibres such as a fabric or other textiles, but may also consist of any type of non-woven structure on which the laden microspheres can be disposed and fixed by thermal application. It is also possible to put into effect the method of the present invention on a substrate not having a fibrous structure but of which the surface finish or external structure allows the distribution and fixing of the microspheres, such as a porous structure. Preferably, the flexible material is selected from woven or non-woven textiles composed of natural fibres of animal or vegetable origin, or synthetic fibres.

The whole of the flexible material including the laden microspheres is then heated to a temperature higher by 1 to 10° Celsius than the melting temperature of the porous crosslinked polymer constituting the microspheres. In a surprising manner, it was observed that it was possible to fix the microspheres to the fibres or pores of the substrate by thermal application, in a precise range of temperatures, which depends on the polymer constituting the microsphere, without damaging the porous crosslinked structure of the microsphere, the molten state of the polymer not being reached. Specifically, this step is carried out in a sufficiently short time to preserve the physicochemical properties of the porous crosslinked polymers used in the preparation of the microspheres and to allow the diffusion of the active substances through the interconnected microporous network of the microspheres. Preferably, its duration is between 1 to 10 seconds. The presence of the active substance within the microspheres appears to play a role in fixing the microspheres on the fibres or pores of the substrate at precise temperatures and in a given time without the molten state of the polymer being reached, the active substance lowering the general viscosity of the laden microsphere.

If several types of polymers are used in the preparation of the microspheres, several heating steps may be provided in the coating process. Each step is adjusted to a temperature higher by 1 to 10° Celsius than the melting temperature of the porous crosslinked polymer in question. In this case the polymers having the highest melting point are processed first and those whose melting point is the lowest are processed last so as not to damage their structure by melting. This makes it possible in particular to combine the properties of more or less slow or fast release of the polymers used in the manufacture of the different microspheres.

The heating can be provided by any type of apparatus used for industrial production of coated flexible materials, especially ovens, heating units with application rollers, or presses. If a pressure force is exerted in conjunction with heating, the heating time and the temperature used are reduced. In this case it is even possible to accomplish the fixing of the microspheres in a very short time, referred to as the “flash time”, and at temperatures below the melting temperature of the polymer, which is of great advantage in terms of preserving the properties both of the polymer and of the stored active substance. The final step is to provide cooling of the material covered in laden microspheres. Preferably, this will be accomplished by means of cooling apparatus provided for this purpose.

The present invention makes it possible to obtain flexible materials such as fibrous or porous substrates laden with one or more active substances that will diffuse into the user's immediate environment in a continuous manner with peak effects extended by sustained diffusion depending on the polymers selected to form the microspheres. No auxiliary product is used thereby ensuring the greater comfort and health of the user. Flexible materials manufactured by the method according to the invention can contain one or more layers of laden microspheres so as to combine the effects of several active substances. These materials can take the form of entire garments that can be worn by the user in direct contact with the skin, such as under-garments, stockings, girdles, belts or vests for example, but also any type of textile or porous accessories that are capable of being worn by the user. They can also include parts of clothing only so as to obtain a diffusion more localised to certain areas of the body. The garments in question can be used for the diffusion of active substances, in direct contact or otherwise with the user's skin.

The following examples are given only by way of illustration of the present invention and should not be interpreted in a restrictive manner as to its scope.

### EXAMPLES

### Preparation of Microspheres

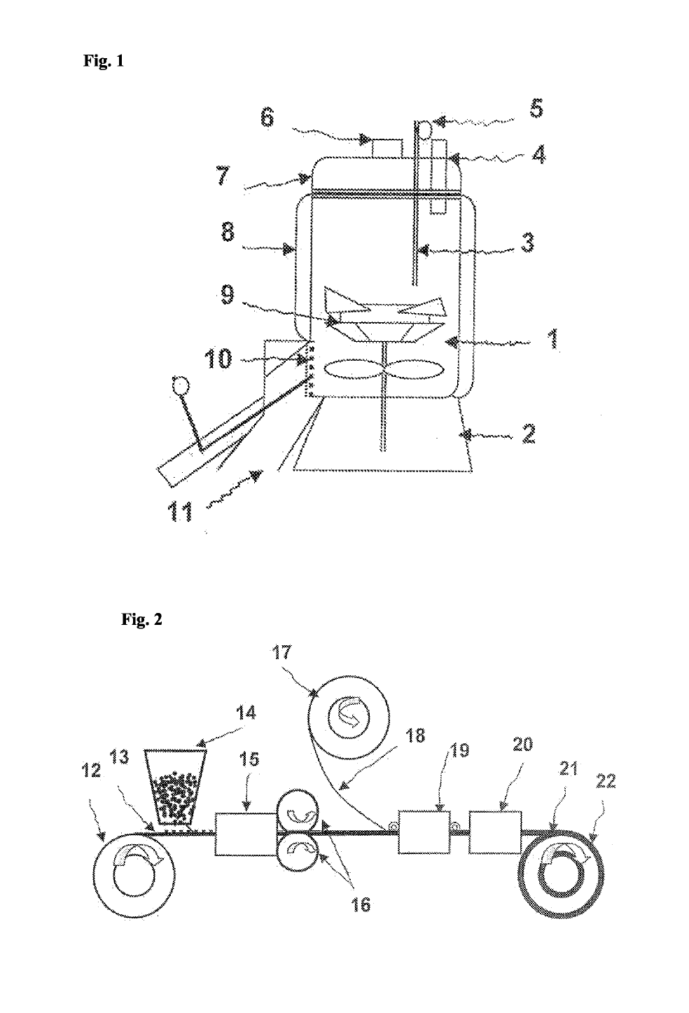

The microspheres are prepared in an apparatus such as that shown in FIG. 1, consisting of a mixing vessel 1 mounted on a base 2, a temperature probe 3, a liquid feed device 4, a thermometer 5 connected to the probe 3, a viewing port 6 disposed on the vessel cover 7, a heating jacket 8, a stirrer system 9, and a discharge port 10 to collect the microspheres presented at the discharge chute 11.

The active substances are charged into the various polymers using technology well known to the applicant (FR 2 901 172, AB7 Industries).
Micronised granules (5 to 200 μm in diameter) of polyamide (ORGASOL), polyether block amides (PEBAX), ethylene and vinyl acetate copolymer (EVA) or other porous crosslinked polymers are charged with active ingredients by incorporation at low temperatures between 20 and 95° C., preferably 70° C.
This granular material or so-called carrier polymer (microsphere), i.e. that in which the active ingredients are incorporated, must be from the family of “porous” crosslinked polymers capable of storing the lipophilic liquid active ingredients by sequestration. The polymer must be solid at room temperature and must have a glass transition temperature (Tg) of between 30 and 95° C. Also its melting point must be between 75 and 180° C., but preferably below 140° C. This polymer must be capable of releasing almost all of the active ingredients incorporated therein.
The active ingredients or solutions of active ingredients and excipients must be lipophilic in nature and can represent up to 40% by weight of the finished product. They are natural essential oils, natural or synthetic essences, natural or synthetic substances in liquid form or dissolved in a lipophilic medium, medicines, perfumes, etc.
The active ingredients are incorporated into the polymer by thermal means, i.e. at 1° C. to 5° C. above the glass transition temperature of the polymer, preferably 3° C. This is done in a short time of less than 5 minutes, preferably 3 minutes, without the use of technical plasticising additives. Cooling completes the incorporation process and ensures the stability of the incorporated active ingredients.
The operation of making the laden microspheres takes place as follows:
   into the bowl 1 of a high-speed laboratory mixer (compounder) such as the model GUNTHER PAPENMEIER KG, having a useful capacity of 8 litres, is placed the quantity of polymer to be charged, pre-heated to the working temperature by hot water or other fluid circulating through the jacket 8;
   the cover 7 is closed to commence the heating process for incorporating the polymer, with stirring by means of the stirrer system 9;
   at fixed temperature and with continued stirring, the liquid active ingredients are introduced via the feed device 4 for 2 to 5 minutes;
   the reaction temperature is maintained for 3 minutes before cooling via the jacket 8 with continued stirring;
the discharge port 10 is then opened to facilitate collection of the laden microspheres via the discharge chute 11.
   Coating of Microspheres onto the Flexible Material

The coating is carried out in an apparatus such as that shown in FIG. 2, consisting of a dispensing roll 12, the flexible material to be treated being referenced 13, a hopper 14 containing microspheres, a heating unit 15, pressing rollers 16, another dispensing roll 17, the flexible material to be applied to the coating being referenced 18, a heating unit with applicator rolls 19, a cooling unit 20, the coated material being referenced 21, and a winding roll 22 for the coated material.

The coating process takes place as follows:

A. transfer of the microspheres (˜5 to 200 μm in diameter) from the hopper 14, freely or in a mould, onto the flexible material 13 reeled off the spool 12;
B. heating in the heating unit 15 to the temperature corresponding to the matrix of the microspheres;
C. pressing of the coating onto the material by means of the pressing rollers 16;
D. application (optionally) of the 2^{nd} flexible material 18 reeled off the spool 17 onto the coating in the heated applicator device 19;
E. cooling of the assembly in the cooling unit 20;
F. winding of the coated material 21 onto the spool 22.

The coated material can be shaped in various ways:

a. by simple cutting or by cutting and stitching;
b. by applying a barrier film on the coated surface to cause the active ingredient to be released from the other face, before cutting or cutting and stitching. Specifically, provision can be made so that it is not the coated face that is in contact with the skin, but the other face, thereby avoiding placing the polymer in direct contact with the skin for sensitive areas of the body such as the face;
c. the coated surface can also be covered by textile.

### Example 1

Tests involving the coating of active polymer in the form of microspheres onto textile were carried out to verify the release of the active ingredient.

The active ingredient was a relaxing fragrance ELISIA supplied by ROBERTET, incorporated into microspheres of polyamide ORGASOL 2002 EXD NAT 1 supplied by ARKEMA, having a melting point of 177° C., according to the applicant's method described hereinabove.

The fabric used was woven cotton, stretchable by 180% in one direction and 36% in the other, supplied by the textile manufacturer ROULEAU GUICHARD. To facilitate the determination of additions and losses, we applied large amounts of active microspheres:
The microspheres were loaded to 25% with ELISIA perfume, a relaxing fragrance,
Pieces of fabric were cut to 20 cm×20 cm,
1 g of microspheres were loaded onto each piece of textile, i.e. 2.5 mg/cm² containing 0.625 mg of perfume,
Parchment paper was placed over the microspheres spread on the textile,
In the absence of a heating apparatus with pressing rollers, an iron at 180° C. was applied on the parchment paper for 4 seconds. Thermal bonding of the microspheres to the textile was obtained without actual melting of the microspheres, and without the addition of any coating aid. The microspheres were bonded to each other and to the fibres. Depending on the amount spread over the fabric, they can form an invisible layer or a flexible visible layer.
In this case, the textile coated with a flexible layer of microspheres laden with Elisia perfume underwent observation of the evaporative capacity of the perfume at room temperature and at 40° C. in an oven. We observed the following:
At room temperature there was an average release of perfume of 2% per day.
At 40° C. in an oven the average release of perfume was 4% per day.

We conclude not only that the release of active ingredient is not impaired, but also that the behaviour of the polymer is not modified in relation to temperature. Heating to 40° C. has the effect of dilating the pores thereby allowing a higher release of perfume as the micro-network of these pores is not severely disrupted by the method of applying the microspheres.

Unlike the current coating process which requires the use of a coating polymer (binder) in which the active microcapsules are trapped, which impairs the release of the active ingredient, the method of the present invention provides a much higher yield. This impairment of the release of the active ingredient is also found in the case of inclusion processes. Only ion bonding appears to come close to these results, but this system presents the difficulty of negatively charging the textile and positively charging the microcapsules, contrasting with the simplicity of the method according to the present invention.

### Example 2

In this example we present the case of an active ingredient that requires a certain formulation before preparing the microspheres. This involves the use of caffeine that we wish to apply topically to obtain a slimming, draining and lipolytic action. This substance has the disadvantage of being only very slightly soluble. It recrystallises as it dries out, and is no longer able in this case to pass through the dermal barrier. To achieve this, the caffeine is formulated with a dispersant, an emulsifier, an emollient and water. The quantity of caffeine in the total formulation is 3%. These formulation substances present no drawbacks of a health-related or toxicological nature as they are authorised and used routinely in the preparation of treatment products. Two polymers were used separately for preparing the microspheres, with a view to comparing their performance. Firstly ORGASOL 2002 EXD NAT 1 supplied by ARKEMA, a polyamide which gives microspheres between 8 μm and 12 μm in diameter and having a melting point of 177° C., and secondly EVA ALCUDIA PA-541 supplied by REPSOL YPF, an ethylene and vinyl acetate copolymer which gives microspheres of 80 μm in diameter and having a melting point of 85-90° C.

We took a 750 cm² piece of textile on which were spread 15,000 mg of laden microspheres, i.e. 20 mg/cm². The coating was applied as described in Example 1 with the application conditions: 180° C. for 4 seconds for the ORGASOL, and 100° C. for 2 seconds for the EVA.

To speed up the release of the active ingredient and for comparison purposes only, we washed the samples as follows:
pieces of each fabric sample were taken;
a water and detergent based washing fluid was prepared in closed test tubes;
each sample piece was immersed in a test tube (3 different pieces per sample), sealed and placed in a rotating carousel chamber to agitate the specimens;
once the chamber was full, it was started and the temperature was raised to 30° C. for 30 minutes;
at the end of the cycle, the test tubes were removed, shaken vigorously (10 back-and-forth strokes by hand), the washing liquid was emptied and replaced with clean water, agitated and rinsed 4 times;
the specimens were removed, air dried and weighed;
the caffeine remaining on the sample was extracted for determination of the quantity by chromatography.

The comparative rate of release of caffeine was 63% for the EVA and 95% for the ORGASOL. This led us to draw the following conclusions:
Microspheres with a high melting point are the ones which release the most active substance, having undergone less structural transformation during the coating process due to the small difference between their melting point and the application temperature.
Polymers are more reactive when their matrix microstructure composed of a micro-network of pores is not disrupted.
The system of coating microspheres without actually melting the material has a major advantage over conventional methods of coating which involve complete melting of the polymer or bonding of a polymer film obtained by extrusion, therefore by complete melting. The melted polymer has a much lower release capacity due to the organisation of its microstructure which provides little continuity of the network of micropores.

### Example 3

EVA microspheres laden with caffeine, as previously described in Example 2, were applied to an extensible textile, as previously described in Examples 1 and 2, for use as a slimming belt (via a lipolytic and/or draining action). The belt measured 120 cm long and 15 cm wide, i.e. having a surface area of 1800 cm². The microspheres were thermally bonded at the rate of 19 mg/cm², or 0.57 mg/cm² of caffeine. A temperature of 100° C. was applied for 2 seconds, so that the melting temperature of 84° C. was not actually reached within the mass of the polymer. Three test cases were conducted:
With the belt worn directly against the skin for 20 days,
With the belt worn over clothing without skin contact for 20 days,
With the belt not worn for 20 days.

These tests revealed that:
The wearer of the belt lost 3 to 4 cm in waist circumference.
The belt worn for 20 days against the skin lost an average of 55% of caffeine, i.e. an average of 0.04 mg/cm² per day. This figure combined with the previous one indicates that caffeine was in fact released to perform its action.
The belt worn over the clothing without contact with the skin showed a 5% loss of caffeine. The latter loss was undoubtedly due mainly to friction.
The belt not worn and left in the open air lost no caffeine but instead gained moisture.

This coating method preserves both the characteristics of the textile and the proper functioning of the microspheres as reservoirs of active ingredients that can be released in a controlled manner.

### Example 4

From the textile samples coated with EVA microspheres laden with caffeine as described in Example 3 were cut 20 cm² pieces which were applied as patches with the aid of adhesive film.

The observation made over a 20 day period under the same analytical conditions as in Example 3 was that 79% of the caffeine was released by the device, i.e. 24% more for the same surface area as the belt.

The fact that the external face is protected by a film that allows the transfer in one direction promotes and accentuates the transfer of caffeine from the coating to the skin.

### Example 5

In this example the microspheres used were charged by surface adsorption of a cosmetic-level active mixture consisting of caffeine, a cosmetic solvent, a cosmetic emulsifier, a cosmetic gelling agent and a cosmetic emollient, on Rilsan® B powder (polyamide 11) supplied by ARKEMA and having a melting point of 175° C., as follows:
The RILSAN® (68%) and CARBOPOL ULTREZ 21 (1%) were preheated in the compounder,
The CAFFEINE (10%) was dissolved at 70° C. in Water (5%), Glycerol (5%), EUMULGIN SMS 20 (1%) and EUTANOL G (10%),
The hot solution was poured into the compounder,
The resultant powder of active microspheres was cooled and removed.

The dry active microspheres were placed between two layers of cotton which were bonded by hydrovaporisation and thermopressing between 120° C. and 150° C., preferably 130° C. for 1 second.

This so-called active cotton was then prepared in the form of squares or rounds. This material was used by applying it to the skin after spraying with a cleansing lotion which instantly dissolved the active mixture adsorbed on the RILSAN® B thereby transferring it to the skin.

This method of applying the active ingredient would not have been effectively accomplished had we used a normal coating process.

1- Method for coating polymer microspheres laden with one or more active substances onto a flexible material without melting, characterised in that it comprises the following steps:
a) selecting a porous crosslinked polymer,
b) incorporating a liquid lipophilic composition at room temperature containing at least one active substance into the polymer by heating at a temperature higher by 1 to 5° C. than the glass transition temperature of the polymer in order to obtain laden microspheres,
c) placing the laden microspheres on a flexible material,
d) heating the flexible material with the laden microspheres at a temperature higher by 1 to 10° C. than the polymer melting temperature, without reaching the molten state of the polymer,
e) cooling the flexible material impregnated with the laden microspheres.

2- Method according to claim 1, characterised in that the crosslinked polymer is chosen from polyamides, polyether block amides or ethylene vinyl acetates.

3- Method according to claim 1, characterised in that a plurality of microspheres composed of different porous crosslinked polymers selected from polyamides, polyether block amides or ethylene vinyl acetates are formed and mixed during step b).

4- Method according to claim 1, characterised in that the active substance is selected from compounds having an effect of a therapeutic, dermatological, cosmetic or olfactory nature.

5- Method according to the preceding claim, characterised in that the active substance is selected from molecules with analgesic, anti-inflammatory, veinotonic, slimming-lipolytic and/or draining, moisturising, soothing, anti-ageing-antioxidant and/or restructuring and/or repairing, clarifying, heavy legs, anti-stress, deodorising/odorising, attractant and/or repellent action.

6- Method according to claim 1, characterised in that the lipophilic composition that is liquid at room temperature is formulated from essential oils, natural or synthetic essences, natural or synthetic substances in liquid form or dissolved in a lipophilic medium.

7- Method according to claim 1, characterised in that step b) is performed in a time of less than 5 minutes.

8- Method according to claim 1, characterised in that, at step b), the active substance is incorporated into the porous cross-linked polymer in a weight ratio representing 1 to 40% by weight of the resulting laden microsphere.

9- Method according to claim 1, characterised in that, at step b), several active substances of different nature are incorporated into one type of microsphere.

10- Method according to claim 1, characterised in that the flexible material comprises a fibrous or porous substrate on which the microspheres are capable of being disposed and fixed by thermal application without the microspheres reaching the melted state.

11- Method according to the preceding claim characterised in that the flexible material is selected from woven and non-woven textiles composed of natural fibres of animal or vegetable origin, or synthetic fibres.

12- Method according to claim 1, characterised in that step d) is performed in a time of between 1 and 10 seconds.

13- Method according to claim 1, characterised in that step d) includes several heating steps adjusted according to a temperature higher by 1 to 10° C. than the melting temperature of the porous cross-linked polymer in question, starting with the highest melting temperatures and ending with the lowest melting temperatures, without in so doing reaching the melted state of the microspheres.

14- Method according to claim 1, characterised in that a pressure force is exerted in conjunction with the heating during step d).

15- Flexible material coated with polymer microspheres laden with one or more active substances obtained according to claim 1, characterised in that it is presented in the form of garments, under-garments or textile or porous accessories that can be worn by a user.

16- Use of a flexible material according to the preceding claim for the controlled diffusion of active substances in direct contact or otherwise with the skin of a user.

17. Flexible material coated with polymer microspheres laden with one or more active substances obtained according to claim 2, characterised in that it is presented in the form of garments, under-garments or textile or porous accessories that can be worn by a user.

18. Flexible material coated with polymer microspheres laden with one or more active substances obtained according to claim 3, characterised in that it is presented in the form of garments, under-garments or textile or porous accessories that can be worn by a user.

19. Flexible material coated with polymer microspheres laden with one or more active substances obtained according to claim 4, characterised in that it is presented in the form of garments, under-garments or textile or porous accessories that can be worn by a user.

20. Flexible material coated with polymer microspheres laden with one or more active substances obtained according to claim 5, characterised in that it is presented in the form of garments, under-garments or textile or porous accessories that can be worn by a user.
